(19) United States
(12) Patent Application Publication
(10) Pub. No.: US 2026/0256611 A1
(43) Pub. Date: Sep. 3, 2026

(54) EXTERNAL NON-INVASIVE URINARY CATHETER FOR MALE PATIENTS

(71) Applicant: Keegan Sauer, Thousand Oaks, CA (US)

(72) Inventor: Keegan Sauer, Thousand Oaks, CA (US)

(21) Appl. No.: 19/653,464

(22) Filed: Apr. 21, 2026

Related U.S. Application Data
(63) Continuation-in-part of application No. 18/206,308, filed on Jun. 6, 2023, now abandoned.

(51) Int. Cl. A61F 5/453

(52) U.S. Cl. CPC A61F 5/453

(57) ABSTRACT

An external urinary fluid management system comprising a tapered body defining a collection region, a circumferential interface and suction region, a proximal inflatable anatomical retention interface, an air inlet region isolated from suction to manage airflow, and a rotatable distal connector configured to couple to a suction source.

### REFERENCE TO PRIOR APPLICATION

This application is a continuation-in-part application of application Ser. No. 18/206,308 filed Jun. 6, 2023, entitled EXTERNAL NON-INVASIVE URINARY CATHETER FOR MALE PATIENTS by Keegan Sauer.

### BACKGROUND OF THE INVENTION

### Field of the Invention

The present invention relates generally to the field of catheters, and more particularly toward an external, non-invasive catheter for use with male patients.

### Description of the Prior Art

A urinary catheter is a hollow, partially flexible tube that collects urine from the bladder and leads to a drainage bag. Urinary catheters come in many sizes and types.

The instant invention seeks to provide for an improved external, non-invasive catheter for male patients.

### SUMMARY OF THE INVENTION

The primary embodiment of the instant provides for A non-invasive urine management device with inflatable retention and internal fluid containment features comprising: a main cylindrical body, said main cylindrical body having an exterior surface, an interior surface, a closed end and an open end wherein said main cylindrical body tapers to a conical configuration at said closed end; a circumferential interface and suction region at said open end; an air inlet region on said exterior surface of said main cylindrical body; a rotatable distal connection proximate said closed end, said rotatable distal connection configured to couple to suction tubing; an inflation actuator proximate said open end wherein said inflation actuator is configured to introduce or remove fluid or air from an inside portion of said main cylindrical body via connection to an inflation mechanism such as a bulb or syringe; a pressure limiting mechanism proximate said inflation actuator configured to vent to atmospheric pressure when a predetermined pressure threshold is reached; and a mesh lining on said interior surface of said main cylindrical body.

### BRIEF DESCRIPTION OF THE DRAWINGS

FIG. 1 is a side perspective view of the catheter of the instant invention.

### DETAILED DESCRIPTION OF A PREFERRED EMBODIMENT

Turning to the drawings, the preferred embodiment is illustrated and described by reference characters that denote similar elements throughout the several views of the instant invention.

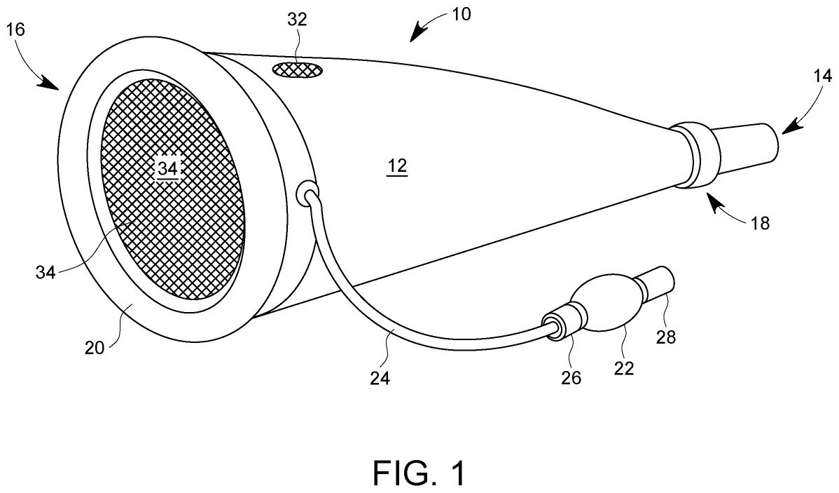

The preferred embodiment of the instant invention is illustrated in in its entirety in FIG. 1.

The catheter 10 is an external non-invasive urinary catheter for male patients, providing urinary incontinence control. The catheter 10 will be applied to the male patient and go over the male's genitals/penile shaft through a front opening 16 in the catheter 10. The catheter's 10 interior wall will be lined with an absorbing mesh 34 that extends all the way to the closed end 14 on the inside of a main body 12. The absorbing mesh 34 is provided to absorb urine which can subsequently be suctioned off so there is no leakage or backflow.

Turning to the figure, the catheter 10 has a main tapered cylindrical body 12 with a closed end 14 and an open end 16. The tapered cylindrical body 12 is the main structural body of the system. It is generally cylindrical tapering to a conical configuration at the closed end 14. It defines a urine collection region and houses the circumferential interface region and suction region 30 and the air inlet region 32. It is configured to operate under negative pressure independent of gravity.

Proximate the closed end 14 of the catheter is a rotatable distal connector 18 that is configured to couple to wall-mounted or potable suction tubing. It is rotatable relative to the cylindrical main body 12 and thereby accommodates movement. It also reduces kinking, torque transmission and dislodgement.

Attached proximate the open end 16 is an inflation actuator 22 connected via conduit 24. The inflation actuator 22 is configured to introduce and remove fluid or air from a retention interface 20 which is where the catheter 10 attaches to the genitals/penile shaft of a patient. The inflation actuator 22 may include a bulb, a syringe or other inflation mechanism depending on the embodiment. At the far end of the inflation actuator opposite from the conduit 24 is the inflation port 28 which is configured to receive the inflation device, i.e., the bulb or syringe and may include a luer-type or equivalent connector. The inflation port 28 provides fluid communication with the inflation circuit and is used to inflate and deflate the anatomical retention interface 20.

On the opposite side of the inflation actuator 22 is a pressure-limiting mechanism 26 that is coupled to the inflation circuit and configured to vent to atmospheric pressure when pressure exceeds a predetermined threshold, which thereby limits the pressure applied by the anatomical retention interface 20. It also protects the patient's tissue from over-inflation. It is isolated from urine flow and suction pathways.

The anatomical retention interface 20 is an inflatable and deflatable retention structure positioned to engage the penile shaft proximal to the glans. It applies distributed, non-occlusive contact pressure and wrapped with a compliant mesh covering 34. It is configured to inhibit distal migration of the system. It does not form a circumferential fluid seal and does not rely on adhesive for fixation.

On an exterior portion of the tapered cylindrical main body 12 is an air inlet region 32 that is spatially isolated from the suction region. It permits airflow into the system under negative pressure. It is configured to prevent collapse, occlusion, or excessive tissue loading and may include a liquid-blocking membrane to inhibit urine egress.

At the open end 16 of the catheter 10 is the circumferential interface region and circumferential suction region 30. It is an internal region extending circumferentially around the penile shaft of the user. It includes a non-occlusive mesh or wicking liner 34 and is configured to interface with urine from multiple angular positions. It works in cooperation with circumferential suction to draw urine downstream. It is spatially separated from the air inlet region 32 and retention interface 20

This external catheter is designed ideally used for urinary incontinent male patients, trauma and mentally altered/intoxicated patients, patients who can't walk or aren't allowed to walk due to injury or sickness and for unconscious patients due to sickness or trauma or during surgeries. The instant invention provides an external non-invasive male catheter, and as contemplated will be a “one size fits all” catheter for male patients.

The invention illustratively disclosed herein suitably may be practiced in the absence of any element which is not specifically disclosed herein.

The discussion included in this patent is intended to serve as a basic description. The reader should be aware that the specific discussion may not explicitly describe all embodiments possible and alternatives that are implicit. Also, this discussion may not fully explain the generic nature of the invention and may not explicitly show how each feature or element can actually be representative or equivalent elements. Again, these are implicitly included in this disclosure. Where the invention is described in device-oriented terminology, each element of the device implicitly performs a function. It should also be understood that a variety of changes may be made without departing from the essence of the invention. Such changes are also implicitly included in the description. These changes still fall within the scope of this invention.

Further, each of the various elements of the invention and claims may also be achieved in a variety of manners. This disclosure should be understood to encompass each such variation, be it a variation of any apparatus embodiment, a method embodiment, or even merely a variation of any element of these. Particularly, it should be understood that as the disclosure relates to elements of the invention, the words for each element may be expressed by equivalent apparatus terms even if only the function or result is the same. Such equivalent, broader, or even more generic terms should be considered to be encompassed in the description of each element or action. Such terms can be substituted where desired to make explicit the implicitly broad coverage to which this invention is entitled. It should be understood that all actions may be expressed as a means for taking that action or as an element which causes that action. Similarly, each physical element disclosed should be understood to encompass a disclosure of the action which that physical element facilitates. Such changes and alternative terms are to be understood to be explicitly included in the description.

What is claimed is:

1. A non-invasive urine management device with inflatable retention and internal fluid containment features comprising:
a main cylindrical body, said main cylindrical body having an exterior surface, an interior surface, a closed end and an open end wherein said main cylindrical body tapers to a conical configuration at said closed end;
a circumferential interface and suction region at said open end;
an air inlet region on said exterior surface of said main cylindrical body;
a rotatable distal connection proximate said closed end, said rotatable distal connection configured to couple to suction tubing;
an inflation actuator proximate said open end wherein said inflation actuator is configured to introduce or remove fluid or air from an inside portion of said main cylindrical body via connection to an inflation mechanism such as a bulb or syringe;
a pressure limiting mechanism proximate said inflation actuator configured to vent to atmospheric pressure when a predetermined pressure threshold is reached; and
a mesh lining on said interior surface of said main cylindrical body.
